(19) United States
(12) Patent Application Publication
(10) Pub. No.: US 2020/0093709 A1
(43) Pub. Date: Mar. 26, 2020

(54) POWDER FORMULATION FOR DENTAL COMPOSITE

(71) Applicant: GC CORPORATION, Sunto-gun (JP)

(72) Inventors: Yoshie Nomura, Kasugai-shi (JP); Kenji Kojima, Kasugai-shi (JP); Tatsuya Fujimoto, Kasugai-shi (JP)

(73) Assignee: GC CORPORATION, Sunto-gun (JP)

(21) Appl. No.: 16/571,859

(22) Filed: Sep. 16, 2019

(30) Foreign Application Priority Data
Sep. 26, 2018 (JP) 2018-179945

(51) Int. Cl. A61K 6/083; C08L 33/12

(52) U.S. Cl. CPC A61K 6/083; C08L 33/12

(57) ABSTRACT

Provided is a powder formulation for a dental composite which prevents aggregation, makes it easy to be obtained by a proper amount, and makes it possible to suppress formation of incineration residues after mixed with a liquid formulation for a dental composite to form resin. The powder formulation includes a flowability-improving agent in the form of powder, the flowability-improving agent being comprising polymethyl (meth)acrylate, the flowability-improving agent having a volume median particle diameter of 0.02 μm to 50 μm.

### TECHNICAL FIELD

The present invention relates to a powder formulation used for a dental composite.

### BACKGROUND ART

For example as JP 2007-51116 A, a dental polymerizable resin formed of a powder component and a liquid component is provided.

### SUMMARY OF INVENTION

### Technical Problem

When such a liquid formulation and a powder formulation are used to at last form resin, the powder formulation may aggregate, which makes it difficult to obtain the powder formulation of a proper amount. In addition, burning out the resin leads to a lot of incineration residues, which is problematic. Formation of incineration residues may cause undesired situations when some steps follow later. For example, when a dental material is poured into a space formed after the resin is burned out, incineration residues adhere to the dental material.

An object of the present invention is to provide a powder formulation for a dental composite which prevents aggregation, makes it easy to be obtained by a proper amount, and makes it possible to suppress formation of incineration residues after mixed with a liquid formulation to form resin.

### Solution to Problem

One aspect of the present invention is a powder formulation for a dental composite, the powder formulation comprising: a flowability-improving agent in the form of powder, the flowability-improving agent comprising polymethyl (meth)acrylate, the flowability-improving agent having a volume median particle diameter of 0.02 μm to 50 μm.

The amount of the flowability-improving agent may be 0.1 mass % to 30 mass % on the basis of the total mass of the powder formulation.

The powder formulation may further comprise: a (meth)acrylate polymer in the form of powder, the (meth)acrylate polymer having a volume median particle diameter of more than 50 μm.

The powder formulation may further comprise: a (meth)acrylate copolymer.

### Advantageous Effects of Invention

According to the present invention, aggregation of the contained powder can be suppressed, which makes it possible to weigh a proper amount of a powder formulation for a dental composite, and makes it easy to handle the powder formulation. In addition, when resin formed by mixing this powder formulation with a liquid formulation for a dental composite is burned out, formation of residues can be suppressed.

### DESCRIPTION OF EMBODIMENTS

Embodiments of the present invention will be described.

A powder formulation for a dental composite according to one embodiment of the present invention is a powder formulation for a dental composite which is used for a composite for a dental pattern resin. The composite for a dental pattern resin is formed by mixing this powder formulation for a dental composite with a liquid formulation for a dental composite. Hereinafter each of the powder formulation and the liquid formulation will be described. Hereinafter “(meth)acrylate” means acrylate and/or methacrylate.

[Powder Formulation for Dental Composite]

### <(Meth)Acrylate Copolymer>

The powder formulation for a dental composite of the present embodiment contains powder of a (meth)acrylate copolymer. Containing such a copolymer in an amount of more than 5 mass % on the basis of the total mass of the powder formulation for a dental composite makes it possible to suppress shrinkage of a pattern resin in polymerization. Specific examples of the (meth)acrylate copolymer include copolymers of ethyl(meth)acrylate and methyl(meth)acrylate.

The composition of the (meth)acrylate copolymer is methyl (meth)acrylate in an amount of 10 mass % to 80 mass %, and ethyl (meth)acrylate in an amount of 20 mass % to 90 mass %.

Containing methyl (meth)acrylate in an amount of more than 80 mass % leads to a difficult swell, which easily leads to an irregular shape. Containing ethyl (meth)acrylate in an amount of more than 90 mass % leads to too much fluidity, which leads to a tendency to be difficult to make a shape. The composition thereof is more preferably methyl (meth)acrylate in an amount of 20 mass % to 60 mass %, and ethyl (meth)acrylate in an amount of 30 mass % to 80 mass %; and further preferably methyl (meth)acrylate in an amount of 20 mass % to 40 mass %, and ethyl (meth)acrylate in an amount of 45 mass % to 75 mass %.

The volume median particle diameter (mean particle size, D50) of the (meth)acrylate copolymer is preferably 40 μm to 130 μm. Containing the (meth)acrylate copolymer having a particle size of this range makes it possible to suppress shrinkage of the composite for a dental pattern resin more certainly when this composite is polymerized, and to prevent the undesirable situations of deformation of the shape and difficult removal from a model. When the volume median particle diameter is less than 40 μm, the swelling rate increases, the curing time shortens, and the amount which can be taken at once with a brush-on technique reduces, which leads to a tendency of deteriorated usability. When the volume median particle diameter is more than 130 μm, the effect of suppressing shrinkage of the composite as described above may be smaller.

The volume median particle diameter thereof is more preferably 40 μm to 100 μm, and further preferably 40 μm to 80 μm in view of further suppressing shrinkage of the composite for a dental pattern resin in polymerization-cure.

Here, “volume median particle diameter”, which may be also referred to as a mean particle size or D50, means a particle size when the cumulative volume index is 50%: the cumulative volume index is calculated by cumulating the volume fractions in order of a particle size from small to large. Such a volume median particle diameter can be measured with a laser diffraction and scattering method.

The amount of the (meth)acrylate copolymer contained in the powder formulation for a dental composite for the composite for a dental pattern resin is preferably more than 5 mass % and no more than 98 mass %. This makes it possible to suppress shrinkage of a pattern resin more certainly in polymerization. The amount thereof is more preferably 50 mass % to 95 mass %. This also makes it possible to improve usability.

<(Meth)Acrylate Polymer>

The powder formulation for a dental composite for the composite for a dental pattern resin of the present embodiment may contain powder of a polymer of methyl (meth)acrylate monomers only.

The amount of the powder of this polymer on the basis of the total mass of the powder formulation is preferably 5 mass % to 50 mass %. When the amount is more than 50 mass %, usability may deteriorate.

Concerning the particle size of the powder of this polymer, the volume median particle diameter (mean particle size, D50) thereof is preferably more than 50 μm and no more than 130 μm. When this particle size is more than 130 μm, the composite for a dental pattern resin when polymerization-cured tends to shrink too much.

<Flowability-Improving Agent>

The powder formulation for a dental composite for the composite for a dental pattern resin contains powder of a flowability-improving agent. This makes it possible to prevent powder in the powder formulation for a dental composite from aggregating.

Polymethyl (meth)acrylate is employed as a flowability-improving agent. Using polymethyl (meth)acrylate as a flowability-improving agent makes it possible to suppress residue formation after the composite for a dental pattern resin is burned out. Residue formation indicates that residues will muddy material for a denture base when the material for a denture base is poured into a space formed after the composite for a dental pattern resin is burned out, which will cause undesired situations.

Therefore, using polymethyl (meth)acrylate as a flowability-improving agent makes it possible to prevent not only the powder formulation for a dental pattern resin from aggregating but also residues from forming after the composite for a dental pattern resin is burned out.

The volume median particle diameter (mean particle size, D50) of the flowability-improving agent is preferably 0.02 μm to 50 μm, and more preferably 0.05 μm to 20 μm. When the volume median particle diameter is less than 0.02 μm, the powder formulation for a dental composite is easy to scatter, which may lead to inconvenience on handleability. When the volume median particle diameter is more than 50 μm, the flowability-improving agent adsorbs the circumferences of other polymers, which may make it impossible to prevent aggregation.

The amount of the polymethyl (meth)acrylate fine particles contained in the powder formulation for a dental composite is preferably 0.05 mass % to 30 mass %. When the amount is less than 0.05 mass %, the flowability lowers, which may lead to deteriorated usability and storability. When the amount is more than 30 mass %, the fine particles easily scatter, which may lead to deteriorated usability. The amount thereof is preferably no less than 0.1 mass %, and further preferably no more than 20 mass %. The amount thereof is further preferably no more than 5 mass % especially from the viewpoint that polymethyl (meth)acrylate fine particles of a small particle size are expensive.

<Polymerization Initiator>

The powder formulation for a dental composite for the composite for a dental pattern resin may contain a polymerization initiator. Any polymerization initiator may be suitably selected in accordance with an aspect of polymerization of the monomers to be polymerized. As the preferred aspect, when a monofunctional (meth)acrylate monomer and a polyfunctional (meth)acrylate monomer are radical-polymerizable, such a polymerization initiator that radicals are formed may be employed.

A polymerization initiator may be suitably selected as described above. Examples thereof include acylphosphine oxides and compounds having an azide group such as camphorquinone, benzil dimethylketal, benzil diethylketal, benzil di(2-methoxyethyl)ketal, 4,4′-dimethylbenzil-dimethylketal, anthraquinone, 1-chloroanthraquinone, 2-chloroanthraquinone, 1,2-benzanthraquinone, 1-hydroxyanthraquinone, 1-methylanthraquinone, 2-ethylanthraquinone, 1-bromoanthraquinone, thioxanthone, 2-isopropylthioxanthone, 2-nitrothioxanthone, 2-methylthioxanthone, 2,4-dimethylthioxanthone, 2,4-diethylthioxanthone, 2,4-diisopropylthioxanthone, 2-chloro-7-trifluoromethylthioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethylaminophenyl)ketone, 4,4′-bisdiethylaminobenzophenone, and (2,4,6-trimethylbenzoyl)diphenylphosphine oxide. One of them may be used alone, or two or more of them may be used in combination.

The amount of the polymerization initiator contained in the powder formulation for a dental composite for the composite for a dental pattern resin may be suitably adjusted as necessary, and is preferably 0.1 mass % to 5 mass %. When the amount is less than 0.1 mass %, there is a probability that necessary cure cannot be carried out. When the amount is more than 5 mass %, cure is too fast, which may lead to the limited operation time.

[Liquid Formulation for Dental Composite]

### <Monofunctional (Meth)Acrylate Monomer>

The liquid formulation for a dental composite for the composite for a dental pattern resin contains a monofunctional (meth)acrylate monomer having one polymerizable functional group of a carbon-carbon double bond (monofunctional structure), which is preferably a radical-polymerizable monofunctional (meth)acrylate monomer.

The amount of a monofunctional (meth)acrylate monomer contained in the liquid formulation for a dental composite for the composite for a dental pattern resin is preferably 65 mass % to 97 mass %. Containing a monofunctional (meth)acrylate monomer as the major component (more than 50 mass %) as described above makes it possible to suppress a temperature rise in polymerization-cure, to avoid the problem of a burn and air bubbles mixing during operation, and to prevent usability from deteriorating. The amount thereof is more preferably no less than 75 mass %. When the amount of a monofunctional (meth)acrylate monomer is more than 97 mass/%, the composite for a dental pattern resin hugely expands due to heating when burned, which may lead to destruction of, and cracks in an investment (refractory material).

Any monofunctional (meth)acrylate monomer may be employed. One monofunctional (meth)acrylate monomer may be used alone, and a plurality of monofunctional (meth)acrylate monomers may be used in combination. Specific examples of a material thereof include methyl (meth)acrylate, isobutyl (meth)acrylate, benzyl (meth)acrylate, lauryl (meth)acrylate, 2,3-dibromopropyl (meth)acrylate, 2-hydroxyethyl (meth)acrylate, 6-hydroxyhexyl (meth)acrylate, 10-hydroxydecyl (meth)acrylate, propylene glycol mono(meth)acrylate, glycerin mono(meth)acrylate, erythritol mono(meth)acrylate, N-methylol (meth)acrylamide, N-hydroxyethyl (meth)acrylamide, N-(dihydroxyethyl) (meth)acrylamide, (meth)acryloyl oxydodecylpyridinium bromide, (meth)acryloyl oxydodecylpyridinium chloride, (meth)acryloyl oxyhexadecylpyridinium chloride, (meth)acryloyl oxydecylammonium chloride, ethyl (meth)acrylate, isopropyl (meth)acrylate, 3-hydroxypropyl (meth)acrylate, 2-hydroxy-1,3-dimethacryloxypropane, t-butyl (meth)acrylate, n-butyl (meth)acrylate, isobutyl (meth)acrylate, butoxyethyl (meth)acrylate, glycidyl (meth)acrylate, 2-methoxyethyl (meth)acrylate, 2-hexylethyl (meth)acrylate, benzil (meth)acrylate, and ethylene glycol (meth)acrylate.

Among them, a methyl (meth)acrylate monomer is preferably employed as a monofunctional (meth)acrylate monomer. According to this, air bubbles are hard to form, and good usability is achieved more certainly.

<Polyfunctional (Meth)Acrylate Monomer>

The liquid formulation for a dental composite for the composite for a dental pattern resin contains a polyfunctional (meth)acrylate monomer having at least two polymerizable functional groups of a carbon-carbon double bond (polyfunctional structure), which is preferably a radical-polymerizable polyfunctional (meth)acrylate monomer.

The amount of a polyfunctional (meth)acrylate monomer contained in the liquid formulation for a dental composite for the composite for a dental pattern resin is preferably 2 mass % to 30 mass %. This makes it possible to suppress expansion of the composite for a dental pattern resin when this composite is burned and heated, and to prevent an investment (refractory material) from being destroyed and cracking. This is imagined to be because a cross-linked structure is formed by polymerization of polyfunctional (meth)acrylate monomers, which suppress expansion even in heating.

When the amount of a polyfunctional (meth)acrylate monomer is less than 2 mass %, the effect of suppressing expansion may be insufficient. When the amount of a polyfunctional (meth)acrylate monomer is more than 30 mass %, the amount of the monofunctional (meth)acrylate monomer is interrelatively reduced, which leads to a high probability of a temperature rise when the composite for a dental pattern resin is polymerization-cured, air bubbles mixing, and deteriorated usability. The amount thereof is more preferably no more than 15 mass %.

Any polyfunctional (meth)acrylate monomer may be employed. One polyfunctional (meth)acrylate monomer may be used alone, and a plurality of polyfunctional (meth)acrylate monomers may be used in combination.

Examples of a polyfunctional (meth)acrylate monomer having two functional groups include ethylene glycol di(meth)acrylate, triethylene glycol di(meth)acrylate, propylene glycol di(meth)acrylate, neopentyl glycol di(meth)acrylate, 1,6-hexanediol di(meth)acrylate, 1,10-decanediol di(meth)acrylate, 2,2-bis[4-{3-(meth)acryloyloxy-2-hydroxypropoxy}phenyl]propane, 2,2-bis[4-(2-(meth)acryloyloxyethoxy)phenyl]propane, 2,2-bis[4-(meth)acryloyloxypolyethoxyphenyl]propane, 1,2-bis[3-(meth)acryloyloxy-2-hydroxypropoxy]ethane, pentaerythritol di(meth)acrylate, and [2,2,4-trimethylhexamethylene bis(2-carbamoyloxyethyl)]di(meth)acrylate.

Examples of a polyfunctional (meth)acrylate monomer having at least three functional groups include trimethylolpropane tri(meth)acrylate, trimethylolethane tri(meth)acrylate, tetramethylolmethane tri(meth)acrylate, pentaerythritol tetra(meth)acrylate, dipentaerythritol hexa(meth)acrylate, N,N′-(2,2,4-trimethylhexamethylene) bis[2-(aminocarboxy)propane-1,3-diol]tetramethacrylate, and 1,7-diacryloyloxy-2,2,6,6-tetraacryloyloxymethyl-4-oxyheptane.

Among them, an ethylene glycol di(meth)acrylate monomer is preferable as a polyfunctional (meth)acrylate monomer having two functional groups, and a trimethylolpropane tri(meth)acrylate monomer is preferable as a polyfunctional (meth)acrylate monomer having three functional groups. According to this, further good physical properties and usability are achieved.

Only one of the polyfunctional (meth)acrylate monomer having two functional groups and the polyfunctional (meth)acrylate monomer having three functional groups may be contained, or both of them may be contained. The polyfunctional (meth)acrylate monomer having three functional groups is preferably contained in view of more effective achievement of the effect of suppressing expansion due to heating in burning. From such a viewpoint, the amount of the polyfunctional (meth)acrylate monomer having three functional groups in all the polyfunctional (meth)acrylate monomers is preferably more than 0 mass % and no more than 20 mass %. When the amount thereof is more than 20 mass %, shrinkage of the composite for a dental pattern resin in polymerization-cure tends to be too much.

<Polymerization Accelerator>

The liquid formulation for a dental composite for the composite for a dental pattern resin may contain a polymerization accelerator. This makes it possible to further suppress expansion when the composite for a dental pattern resin is heated to be burned out, and to prevent an investment (refractory material) from being destroyed and cracking. It is imagined to be because polymerization is more certainly carried out with a polymerization accelerator, which makes it possible to reduce unpolymerized monomers.

A specific material as a polymerization accelerator may be suitably selected, and a tertiary amine and the like may be mainly employed. Examples of a tertiary amine include N,N′-dimethyl-p-toluidine, N,N′-dimethylaminoethyl methacrylate, triethanolamine, 4-dimethylamino methyl benzoate, 4-dimethylamino ethyl benzoate, and 4-dimethylamino isoamyl benzoate. Other examples include benzoyl peroxide, sodium sulfinate derivatives, and organometallic compounds. One of them may be used alone, or two or more of them may be used in combination.

Among them, N,N′-dimethyl-p-toluidine is preferable. This makes it possible to efficiently accelerate polymerization, and to make the above described effect greater.

The amount of the polymerization accelerator contained in the liquid formulation for a dental composite for the composite for a dental pattern resin is preferably 3 mass % to 30 mass %. This makes it possible to achieve the above described effect more certainly. When the amount thereof is less than 3 mass %, there is a probability of insufficiently achieving the effect as a polymerization accelerator. When the amount thereof is more than 30 mass %, storage stability tends to deteriorate.

<Others>

Other UV absorber, coloring agent, and polymerization inhibitor may be contained as necessary. Any known ones may be employed for them.

[Aspect of Composite for Dental Pattern Resin]

The composite for a dental pattern resin is formed by mixing the liquid formulation for a dental composite and the powder formulation for a dental composite. The proportions of incorporating the liquid formulation for a dental composite and the powder formulation for a dental composite may be suitably set as necessary. The composite for a dental pattern resin is preferably constituted so as to be usable without any problem as containing the liquid formulation for a dental composite in an amount of 23 mass % to 43 mass %, and the powder formulation for a dental composite in an amount of 57 mass % to 77 mass % on the basis of the total mass of the composite for a dental pattern resin. In many cases, “liquid formulation for a dental composite:powder formulation for a dental composite=1 ml:2 g” is the standard.

In such a composite for a dental pattern resin, containing the flowability-improving agent in the powder formulation for a dental composite which forms the composite for a dental pattern resin can prevent the powder formulation for a dental composite from aggregating, which makes it possible to obtain the powder formulation for a dental composite of a proper amount.

Combination with the above described other materials makes it possible to prevent shrinkage in polymerization and expansion in heating in burning as usability for molding is kept in a process of dental precision casting including the steps of molding, polymerization, and burning. Therefore, the composite for a dental pattern resin can have high usability with high accuracy.

The flowability-improving agent, which is polymethyl (meth)acrylate, makes it possible to further prevent formation of residues after burning as well.

In particular, containing a polyfunctional monomer in the liquid formulation for a dental composite in order to prevent such expansion tends to lead to much shrinkage of the liquid formulation for a dental composite in polymerization. Against this, employment of the powder formulation for a dental composite makes it possible to prevent this shrinkage, and to make the composite for a dental pattern resin have a comprehensively high function.

[Production Method]

The composite for a dental pattern resin can be produced as follows, for example:

Materials of the powder formulation for a dental composite are weighed, and mixed and stirred using a mixer, a mortar, a bag, or the like. At this time, an organic solvent or the like may be added thereto for fixing a pigment. Water and alumina balls may be added as well for improving stirring efficiency. After stirred, the obtained powder may be put through a sieve. A step of crushing the flowability-improving agent in advance may be included.

Materials of the liquid formulation for a liquid formulation are weighed, and mixed and stirred using a mixer or the like.

[Powder Formulation for Dental Composite of Other Aspects]

Here, the powder formulation for a dental composite for a dental pattern resin has been described as one embodiment of employing the powder formulation for a dental composite. The present invention is not limited thereto. The powder formulation for a dental composite may be used as a powder formulation for a dental composite for other resins such as a powder formulation for a dental composite for a resin for a denture base, and a powder formulation for a dental composite for an autopolymerizing resin.

### EXAMPLES

### Example A

In Example A, the powder formulation for a dental composite and the liquid formulation for a dental composite described above were prepared. Aggregation properties of the powder formulation for a dental composite, and discharging performance thereof from a case were evaluated. In addition, the powder formulation for a dental composite and the liquid formulation for a dental composite were mixed to form a dental pattern resin, and incineration residues of this dental pattern resin were evaluated. The subjects of evaluation in Example A were three, that is, Example 1. Comparative Example 1 and Comparative Example 2. Table 1 shows the materials of the powder formulations for a dental composite and the liquid formulations for a dental composite of every Example, and the amounts thereof. The materials are as follows. The units for the numerals in Table 1 are all mass %.
“(Meth)acrylate copolymer” in the powder formulation for a dental composite was a copolymer of ethyl methacrylate and methyl methacrylate, and the composition thereof was ethyl methacrylate in an amount of 70 mass %, and methyl methacrylate in an amount of 30 mass %. The volume median particle diameter was 60 μm.
“(Meth)acrylate polymer” in the powder formulation for a dental composite was polymethyl methacrylate. The volume median particle diameter thereof was 90 μm.
“Flowability-improving agent (PMMA)” in the powder formulation for a dental composite was polymethyl methacrylate. The volume median particle diameter thereof was 0.5 μm.
“Flowability-improving agent (other than PMMA)” in the powder formulation for a dental composite was derived from silica.
“Monofunctional (meth)acrylate monomer” in the liquid formulation for a dental composite was methyl methacrylate.
“Bifunctional (meth)acrylate monomer” in the liquid formulation for a dental composite was ethylene glycol dimethacrylate.
“Trifunctional (meth)acrylate monomer” in the liquid formulation for a dental composite was trimethylolpropane trimethacrylate.
“Polymerization accelerator” in the liquid formulation for a dental composite was a tertiary amine, which was more specifically N,N′-dimethyl-p-toluidine.

Aggregation properties of the powder formulation for a dental composite of each Example, and discharging performance thereof from a case were evaluated. In addition, the powder formulation for a dental composite and the liquid formulation for a dental composite were mixed to form a dental pattern resin, and incineration residues were evaluated. Each evaluation was carried out as follows:

Aggregation properties were evaluated as follows:

A transparent case was filled with the powder formulation for a dental composite, and kept at 45° C. for one week. Thereafter the transparent case that was filled with the powder formulation for a dental composite was shaken ten times, and thereafter turned upside down, to observe how the powder formulation for a dental composite dropped.

Discharging performance from a case was evaluated as follows:

A case with a narrow opening and a cap was filled with the powder formulation for a dental composite, to observe whether powder was able to be continuously discharged from the case without being caught.

Incineration residues were evaluated as follows:

A solid that was the polymerized powder formulation for a dental composite and liquid formulation for a dental composite at the ratio of 2 g:1 ml was put into a crucible, and burned out as raising a temperature from a room temperature to 700° C. After the solid was burned out, the amount of residues was measured.

In every evaluation, when the results were especially better than those of a conventional product (GC PATTERN RESIN by GC Corporation), the evaluation was graded as excellent, when the results were equal to those of a conventional product, the evaluation was graded as good, and when the results were worse than those of a conventional product, the evaluation was graded as bad.

Table 1 shows the results of the evaluation together with the materials of each Example.

TABLE 1

| | | | Comp. | Comp. |
|---|---|---|---|---|
| Item | Material | Ex. 1 | Ex. 1 | Ex. 2 |
| Powder | (Meth)acrylate copolymer | 95 | 95 | 96 |
| formulation | (Meth)acrylate polymer | 4 | 4 | 4 |
| | Flowability-improving agent (PMMA) | 1 | 0 | 0 |
| | Flowability-improving agent (other than PMMA) | 0 | 1 | 0 |
| | Pigment | trace | trace | trace |
| Liquid | Monofunctional (meth)acrylate monomer | 87 | 87 | 87 |
| formulation | Bifunctional (meth)acrylate monomer | 5 | 5 | 5 |
| | Trifunctional (meth)acrylate monomer | 5 | 5 | 5 |
| | Polymerization accelerator (tertiary amine) | 3 | 3 | 3 |
| | UV absorber | trace | trace | trace |
| | Polymerization inhibitor | trace | trace | trace |
| Result | Aggregation properties | excellent | good | bad |
| | Discharging performance from case | excellent | good | bad |
| | Incineration residues | excellent | bad | excellent |

As is seen from Table 1, containing the flowability-improving agent in the powder formulation for a dental composite makes aggregation properties and discharging performance from a case excellent. In addition, using PMMA as the flowability-improving agent makes it possible to suppress residues in burning after curing is performed to form resin.

### Example B

In Example B, Examples 11 to 16 of using PMMA as a flowability-improving agent, and changing the volume median particle diameter thereof were evaluated. Specific material components and evaluation results are shown in Table 2. The contents in Table 2 are the same as in Example A except that the numerals in the flowability-improving agent in “Material” (unit “μm”) represent the volume median particle diameters of PMMA.

TABLE 2

| Item | Material | Ex. 11 | Ex. 12 | Ex. 13 | Ex. 14 | Ex. 15 | Ex. 16 |
|---|---|---|---|---|---|---|---|
| Powder | (Meth)acrylate copolymer | 95 | 95 | 95 | 95 | 95 | 95 |
| formulation | (Meth)acrylate polymer | 4 | 4 | 4 | 4 | 4 | 4 |
| | Flowability-improving agent (PMMA · 0.02 μm) | 1 | 0 | 0 | 0 | 0 | 0 |
| | Flowability-improving agent (PMMA · 0.05 μm) | 0 | 1 | 0 | 0 | 0 | 0 |
| | Flowability-improving agent (PMMA · 0.5 μm) | 0 | 0 | 1 | 0 | 0 | 0 |
| | Flowability-improving agent (PMMA · 5.0 μm) | 0 | 0 | 0 | 1 | 0 | 0 |
| | Flowability-improving agent (PMMA · 20 μm) | 0 | 0 | 0 | 0 | 1 | 0 |
| | Flowability-improving agent (PMMA · 50 μm) | 0 | 0 | 0 | 0 | 0 | 1 |
| | Pigment | trace | trace | trace | trace | trace | trace |
| Liquid | Monofunctional (meth)acrylate monomer | 87 | 67 | 90 | 60 | 40 | 97 |
| formulation | Bifunctional (meth)acrylate monomer | 5 | 15 | 5 | 5 | 30 | 0 |
| | Trifunctional (meth)acrylate monomer | 5 | 15 | 5 | 5 | 30 | 0 |
| | Polymerization accelerator (tertiary amine) | 3 | 3 | 0 | 30 | 3 | 3 |
| | UV absorber | trace | trace | trace | trace | trace | trace |
| | Polymerization inhibitor | trace | trace | trace | trace | trace | trace |
| Result | Aggregation properties | good | excellent | excellent | excellent | excellent | good |
| | Discharging performance from case | good | excellent | excellent | excellent | excellent | good |
| | Incineration residues | excellent | excellent | excellent | excellent | excellent | excellent |

As is seen from Table 2, PMMA having a volume median particle diameter of 0.05 μm to 20 μm leads to especially excellent aggregation properties and discharging performance from a case.

### Example C

In Example C, Examples 21 to 26 of using particles of PMMA whose volume median particle diameter was 0.5 μm as a flowability-improving agent, and changing the amounts thereof were evaluated. Specific material components and evaluation results are shown in Table 3. In this Example, usability was further evaluated. Usability was evaluated with a brush. Specifically, a brush having immersed in the liquid formulation for a dental composite was dipped into the powder formulation for a dental composite, and a mass of the powder formulation for a dental composite was formed at the point of the brush. The mass was taken at mixing paper, and the following were observed: the swelling rates of the powder formulation for a dental composite and the liquid formulation for a dental composite, removability from the brush when the mass was transferred from the brush to the mixing paper, and the shape of a cured product. The evaluation standards are the same as the other evaluation items.

The other contents in Table 3 are the same as in Example A.

TABLE 3

| Item | Material | Ex. 21 | Ex. 22 | Ex. 23 | Ex. 24 | Ex. 25 | Ex. 26 |
|---|---|---|---|---|---|---|---|
| Powder | (Meth)acrylate copolymer | 95.95 | 95.90 | 91 | 86 | 76 | 66 |
| formulation | (Meth)acrylate polymer | 4 | 4 | 4 | 4 | 4 | 4 |
| | Flowability-improving agent (PMMA) | 0.05 | 0.1 | 5 | 10 | 20 | 30 |
| | Pigment | trace | trace | trace | trace | trace | trace |
| Liquid | Monofunctional (meth)acrylate monomer | 87 | 67 | 90 | 60 | 40 | 97 |
| formulation | Bifunctional (meth)acrylate monomer | 5 | 15 | 5 | 5 | 30 | 0 |
| | Trifunctional (meth)acrylate monomer | 5 | 15 | 5 | 5 | 30 | 0 |
| | Polymerization accelerator (tertiary amine) | 3 | 3 | 0 | 30 | 3 | 3 |
| | UV absorber | trace | trace | trace | trace | trace | trace |
| | Polymerization inhibitor | trace | trace | trace | trace | trace | trace |
| Result | Aggregation properties | good | excellent | excellent | excellent | excellent | excellent |
| | Discharging performance from case | good | excellent | excellent | excellent | excellent | excellent |
| | Usability (brush) | excellent | excellent | excellent | excellent | excellent | good |
| | Incineration residues | excellent | excellent | excellent | excellent | excellent | excellent |

As is seen from Table 3, PMMA in an amount of no less than 0.1 mass % as the flowability-improving agent makes aggregation properties and discharging performance from a case excellent. PMMA in an amount of 0.1 mass % to 20 mass % as the flowability-improving agent further makes usability especially excellent.

What is claimed is:

1. A powder formulation for a dental composite, the powder formulation comprising:
a flowability-improving agent in a form of powder, the flowability-improving agent comprising polymethyl (meth)acrylate, the flowability-improving agent having a volume median particle diameter of 0.02 μm to 50 μm.

2. The powder formulation for a dental composite according to claim 1,
wherein an amount of the flowability-improving agent is 0.1 mass % to 30 mass % on the basis of the total mass of the powder formulation.

3. The powder formulation for a dental composite according to claim 1, the powder formulation further comprising:
a (meth)acrylate polymer in a form of powder, the (meth)acrylate polymer having a volume median particle diameter of more than 50 μm.

4. The powder formulation for a dental composite according to claim 1, the powder formulation further comprising: a (meth)acrylate copolymer.
